(19) United States
(12) Patent Application Publication
(10) Pub. No.: US 2005/0175988 A1
(43) Pub. Date: Aug. 11, 2005

(54) Multiple virus reaction assay

(76) Inventors: Stuart M. Wilson, London (GB); Christopher J. Stanley, Woodhurst (GB); Jay Patel, Wembley (GB)

Correspondence Address: Bourque & Associates, Suite 301, 835 Hanover Street, Manchester, NH, 03104, US

(21) Appl. No.: 10/494,896

(22) PCT Filed: Nov. 8, 2002
(86) PCT No.: PCT/GB02/05057

(30) Foreign Application Priority Data
Nov. 9, 2001 (GB) 0126976.0

(51) Int. Cl. C12Q 1/70

(52) U.S. Cl. 435/5

(57) ABSTRACT

A method of assay for the outcome of a reaction comprises conducting a reaction between a first reactant and a second reactant to produce from the first reactant a product having at least one new structural feature not present in the first reactant or the second reactant, e.g. by cleaving the first reactant or adding to it, producing a multiple-phage tagged complex comprising at least two different viruses and said product or said first reactant, in which complex at least one of said viruses is connected to said product using a said new structural feature or is lost from said first reactant upon formation of said new structural feature, and determining the presence or amount of said complex by a dual-phage assay.

The present invention relates to an assay which detects a structural feature of a reaction product and thereby may detect the presence of a reactant, the production of the reaction product, or the effectiveness of a reaction modulator.

WO99/63348 discloses a multiple virus assay in which a reaction product is produced which includes at least two viruses, each of which endows the reaction product with a respective distinctive property. Generally, the viruses are bacteriophage and the distinctive property may be to confer an antibiotic resistance on a suitable bacterium. The chances of a single bacterium becoming infected with two or more different phages are very much increased when the two phages are held in close proximity by being incorporated in the reaction product.

Reaction schemes are described in which two reactants become bound together and are then exposed to two phages. One phage has been given the ability to bind specifically to one of the reactants and the other phage has been given the ability to bind specifically to the other reactant, so that both phages become bound to the reaction product. This is then detectable by cultivation of the reaction product with a suitable bacterium under conditions in which only a bacterium infected by both phages will multiply.

Additionally, reaction schemes are described in which both reactants are prior labelled with respective phages and are then reacted together to form a reaction product in which both phages are retained. The reaction product may then be detected by the multiple phage assay described above.

These methods are especially suitable for monitoring reactions in which first and second reactants form a complex in which both reactants are present.

The degree to which a third reactant modulates (promotes or hinders) the reaction may be observed.

There is also a need to develop improved methods for observing reactions in which a first reactant is modified by reaction with a second reactant other than by forming a complex involving both reactants. For instance a first reactant may be a substrate for an enzyme constituting the second reactant and may be modified by the enzyme which then releases the altered substrate and does not form a permanent complex with it.

There is a particular requirement in the pharmaceutical and agrochemical industries for rapid, high throughput enzyme assays in the field of new chemical entity discovery. High throughput screening systems have been established for the testing of very large libraries of compounds against well characterised enzyme targets, with the aim of identifying novel compounds that modulate the activity of the enzyme. A variety of methods for enzyme assay have been developed that commonly use specific substrates that generate a signal when modified by the enzyme. For enzymes such as hydrolases or proteases this can involve the liberation of a coloured, fluorescent or luminescent moiety on cleavage of the substrate. The signal from the enzyme assay can be monitored by standard instrumentation. For enzymes such as kinases or ligases the product can be detected by fluorescence or radio labelling and carrying out a step to separate substrate from product. Alternatively the products from enzyme reactions can be detected by immunoassay methods using antibodies with specificity for either the substrate or the product.

Some enzymes are of particular importance in the pharmaceutical industry and there is a significant focus on kinases and proteases. These are often involved in metabolic processes that are linked, for example, to proliferation in cancer calls or to viral infection in HIV. U.S. Pat. No. 5,763,198 describes rapid, quantitative assay systems for screening test compounds for their ability to modulate tyrosine kinase or phosphatase activities. A method involving an anti-phoophotyrosine antibody and also an antibody specific for the protein substrate is employed; the assay involves immobilising the protein substrate to a surface prior to detecting the phosphotyrosine moieties on the protein substrate. WO 00125477 discloses methods for screening for modulators of serine or threonine kinase or phosphatase activity that involve the provision of a substrate of the kinase that can be phosphorylated and then contacting the substrate with a reporter which binds the phosphorylated substrate with higher affinity than the unphosphorylated substrate. WO 9009169 discloses novel methods for detecting kinase activity in solution that use a substrate that is phosphorylated in solution to form a product that comprises a phosphorylation independent first tag and a phosphorylation dependent second tag. To complete the assay the product is immobilized onto a solid substrate via a specific binding interaction with a first receptor through the first or second tags. The immobilised conjugate so produced is detected by adding a second receptor to bind the other of the first and second tags, so that the presence of the second receptor indicates the phosphorylation of the substrate and is therefore a measure of kinase activity.

U.S. Pat. No. 5,580,747 describes the assay of kinases, phosphatases and proteases by incubating the enzyme with a substrate modified peptide to form a product modified peptide, separating the two forms of the peptide by chromatography, electrophoresis or extraction and measuring the product modified peptide by, in the case of separation by agarose gel electrophoresis, quantitation of fluorescence from the labelled product modified in the gel.

There is a need for new assay methods for a variety of enzymes and other reactants, that are sensitive, rapid, simple to perform, have the minimum of procedural steps and are suitable for automation in high throughput screening

The present invention now provides a method of assay for the outcome of a reaction comprising conducting a reaction between a first reactant and a second reactant to produce from the first reactant a product having at least one new structural feature not present in the first reactant or the second reactant, producing before or after said reaction a multiple-virus tagged complex comprising at least two different viruses and said product or said first reactant in which complex at least one of said viruses is connected to said product using a said new structural feature or is lost from said first reactant upon formation of said new structural feature, and determining the presence or amount of said complex.

The presence or amount of said complex may be determined by a method comprising exposing to said complex an indicator material to which the viruses carried by the multiple-virus tagged complex attach so as to endow the indicator material with a distinctive property, and observing said distinctive property.

Said indicator material is preferably a bacterial culture and the distinctive property is preferably the ability to survive or multiply under specific culture conditions in which the bacterial culture without said distinctive property does not survive or multiply. Said distinctive property may therefore be multiple antibiotic resistance.

In the method of the invention, two viral particles are physically linked together in the multiple-virus tagged complex and can thus each infect the same bacterial cell so as to endow that cell with both of the characteristic properties of the infecting viruses. A bacterial cell infected by both viruses and possessing the sum of the two characteristic properties can readily be distinguished from cells which possess only one of those properties. For example, the infected cells can be cultivated under conditions under which the cells possessing only one of the properties cannot survive, for example in the presence of specific antibiotics or specific temperature or pH conditions. The infected cells having both characteristic properties survive and will replicate. If lysogenic viruses are used as the tags, the viruses will replicate within the infected bacterial cells and produce progeny virus particles which will be released to begin further cycles of infection and replication. Thus, in the presence of a multiple-virus tagged complex, a cascade of bacterial growth indicates that the complex was present in the initial sample. If no multiple-virus tagged material is present in this cultivation stage, little or no bacterial growth will take place.

Preferably, for detection, said complex is cultured with at least the statistically required amount of bacterial cells.

The term virus is used herein to denote true viruses and organisms which infect bacteria in manner similar to a true virus. Thus, the term virus includes:
a. Components of a virus which have the characteristics of the virus from which they are derived;
b. Packaged phagemids or cosmids, which are crosses between plasmids and viruses and can grow as plasmids in bacterial hosts, but which can be packaged and secreted as if they were viral particles in the presence of a helper virus although they cannot independently produce viral progeny;
c. Viruses which are lysogenic for bacteria and can grow, replicate and produce progeny in the bacteria without lysis of the bacteria which can continue to grow and replicate.

If viral infection of bacterial cells is carried out using an excess of bacteria over that required to achieve parity between the infecting viruses and the infectable bacterial cells, a given bacterium cell is unlikely to be infected by more than one virus particle. The amount at which such dual infection becomes sufficiently unlikely that it will not distort the results of the assay method of the invention can be calculated statistically and is denoted herein as the statistical amount. Such a statistical calculation can be confirmed by simple trial and error tests.

As indicated above, it is necessary for at least two different viruses to become attached to the target material so that cultivation conditions can be selected to ensure that only the indicator material having the properties endowed by both viruses survives. Typically, the viruses used are different species, each imparting a different property to the multiple-virus tagged complex and hence to the indicator material. However, it is within the scope of the present application to use the same species of virus and to modify the virus using known techniques to introduce desirable components not normally present in the virus so as to impart the desired properties to the viruses used in the method of the invention. For example, a virus can be treated in a known manner to introduce a gene which imparts resistance to certain antibiotics. This may be done for each of the viruses which are to be bound in the complex. Such modification can be to introduce other properties into the virus, for example heat or light sensitivity, so that the conditions under which the cultivation are carried out can engender or reflect a wide range of properties in the indicator material.

As indicated above, the virus is bound in the multiple-virus tagged complex so as to endow the complex with at least two different properties. If desired, the complex may be endowed with additional properties carried by the viruses or additional viruses which enable or facilitate detection of the virally attached indicator material. For example a third virus can be bound in the complex which endows the complex with photo-luminescent properties. The presence of the multiple-virus tagged complex can thus be readily detected by illuminating the complex with UV, IR or visible light to cause the material carrying the third virus to illuminate. Alternatively, the property could be the expression of an enzyme not normally expressed by the first or second reactant, for example B-galactosidase, luciferase, or alkaline phosphatase.

Optionally therefore, the multiple-virus tagged complex endows the indicator material with an optically (optionally visually) detectable feature.

The second reactant is preferably an enzyme, e.g. a kinase, phosphatase, adenylating enzyme, ubiqitinylating enzyme, protease, hydrolase, esterase, ligate, polymerase, nuclease, methylase, or glycosidase or other sugar modifying enzyme.

The first reactant may be a substrate cleaved by the second reactant (e.g. enzyme). The new structural feature in this case may be the new terminus provided in the first reactant by the cleavage. It may be that the first reactant is a substrate to which a moiety is added by the second reactant (e.g. enzyme). The new structural feature in this case may be the added moiety. Alternatively, it may be that the first and second reactant form a complex and the new structural feature may be produced by the proximity of features of the first and second reactants at their junction in the complex formed by the first and second reactants. The tagging of the first reactant or of the complex with virus may be carried out in various ways. The binding can be directly to the virus, for example through suitable sites on the surface of the virus particle or through a site which has been modified in a known manner to bind to the reactant or complex. However, it is within the scope of the present invention to bind either or both of the viral particles to the reactant or complex through an intermediate binding material or ligand, for example a protein, amino acid, lectin, peptide, antibody, monoclonal antibody, nucleic acid or other material which recognises different regions of the target material. Alternatively, the genetic sequence encoding the reactant or ligand can be inserted into the virus genome and expressed on the surface of the viral particle. If desired, the site to which a viral particle is to bind can have been flagged with different haptens, for example biotin and dinitrophenol (DNP), and the different viral particle cross-linked with anti-biotin and anti-DNP antibodies so that the viral particles are guided to the binding sites on the target itself or the ligand.

The term binding or tagging of the virus to the reactant or reaction product is therefore used herein to denote attachment of the virus by any means to its target material, whether directly or indirectly, so as to provide sites which retain the activity of the virus operatively associated with the complex.

Suitable pairings of the reactant or reaction product, virus and ligands can readily be established using known techniques and the selections verified by simple trial and error tests. Since the possible combinations of virus and ligand enable a wide range of materials to be formed which can bind to a given target material, and since a wide range of modifications can be achieved to different species of virus, the invention can be used to form a virally bound reactant or complex with a wide range of target materials.

The binding of the virus or modified virus may be carried out using a wide range of methods and materials, depending upon the nature of the target material (reactant or reaction product). Typically, the binding will be carried out by incubating a sample containing the desired target material in the presence of the appropriate viruses, either in a single stage using a mixture of viruses or as two stages where the sites at which the virus particles bind will only accept one type of virus. The incubation is typically carried out at a temperature of from 0 to 60° C. in a suitable liquid medium, notably an aqueous medium. The target material and unbound virus may be in solution, suspension in a liquid phase or may be in solid form or carried upon a solid carrier, for example a nitrocellulose or nylon membrane, a ceramic frit, solid beads or the like. For convenience, the invention will be described hereinafter in terms of the use of an aqueous carrier for the target material. Such a form of the target may be made by dissolving or suspending a freeze dried or other solid form of the virus and target material using known techniques.

The incubation of virus with target material (first reactant or reaction product) is carried out until a satisfactory proportion of the viral particles have become bound to the target material. The optimum conditions for the viral binding stage will depend upon the nature of the target material, the virus particles and the method used to detect the multiple-virus tagged complex. Typically, the viral binding will take from 5 to 180 minutes at a temperature of from near ambient to 60° C., and the optimum period and conditions can readily be determined by simple trial and error tests for any given case. The optimum extent of viral binding to the target material will depend upon the nature of the properties to be detected in the reaction product and the expected level of virally bound target material in the sample, but will typically achieve viral binding of at least 25%, preferably from 30 to 60% or more, of the target material. However, viral binding of 5% or less should be sufficient.

If desired, the virally bound target material can be isolated from the mixture in which it was formed using conventional isolation and washing techniques. Some partial separation of the virally bound target material from residual free virus particles may be carried out using, for example, filtration or capture with paramagnetic beads. The washing may be carried out with materials which kill or incapacitate free viral particles. Alternatively, where the binding of the viral particles to the target material involves labelling the target material with a tag such as biotin, the virally bound target material may be isolated from the unbound viral particles using such a tag, for example by means of streptavidin paramagnetic beads. Such partial separation or isolation of the multiple-virus tagged complex from residual unbound phage enhances the sensitivity and specificity of the method of the invention in determining the presence or amount of the complex.

The first reactant is suitably tagged with a virus prior to reaction with the second reactant and the multiple-virus tagged reaction complex is formed by exposing the product of the reaction between the first and second reactants to a second virus that binds to the said product to form the multiple-virus tagged complex. Alternatively, the first reactant is not virus-tagged and the product of the reaction between the first and second reactants is exposed to a first virus and to at least a second virus which each bind to the said product to form the multiple-virus tagged complex.

The first reactant may be a fusion protein or peptide expressed as part of a coat protein of a phage.

The viruses are each preferably phages. Each of the two or more viruses may be attached to, or may express on their surfaces, specific binding agents such as antibodies. In the case of an assay for kinases it is possible to synthesise a peptide that contains the phosphorylatable residue (tyrosine, serine or threonine) in a specific sequence that is recognised by the kinase. A second moiety such as biotin can also be introduced into the peptide, preferably at some distance from the tyrosine moiety to avoid interference caused by overlap between the structures of the two specific binding agents. It is also possible to synthesise a peptide containing two or more spatially separated tyrosine, serine or threonine residues located in identical sequences that are both phosphorylated by the kinase.

In a preferred embodiment two phage conjugates are employed, the first is conjugated to an antibody with specificity for say phosphotyrosine whilst the second phage is conjugated to an antibody, or other specific binding agent such as streptavidin, with specificity for biotin. On mixing an unmodified peptide conjugated to biotin with the two phage conjugates only one complex will form involving the biotin moiety and the specific binding agent and so, without the second conjugate binding to the peptide substrate, no proximity will be observed. If, however, the tyrosine residue has been phosphorylated by a kinase enzyme prior to incubation with the two phage conjugates then the peptide will bind both conjugates and a significant proximity enhancement of infection of a bacterial cell will take place.

In another preferred embodiment a peptide substrate is used that has two or more identical sites in the amino acid sequence, which can for example be phosphorylation sites. In this embodiment each of two phage conjugates employed has a respective phage conjugated to an antibody with specificity for phospho-tyrosine, serine or threonine. The proximity enhancement effect will only be observed if both residues in the peptide are phosphorylated by the kinase.

The method is not limited to peptide enzyme substrates. For kinase assays the phosphorylatable residue may be on a protein substrate and two or more phages can be employed linked to specific binding agents with specificity for the phosphorylatable residue and for epitopes on the protein itself at different positions around the protein surface. Binding to the phosphorylated residue and the protein will lead to a proximity enhancement effect. The protein can be in solution or it can be immobilized to a surface.

In a further embodiment the protein or peptide sequence containing, for example, a phosphorylatable residue or a cleavage site for a protease can be expressed on the surface of one or more viruses. In the bacteriophage M13, for example, a fusion protein can be created so that the coat protein also contains the specific peptide sequence or protein. In this example the enzyme will phosphorylate, cleave, adenylate etc. the expressed sequence. Then one or more phage linked to binding agents with specificity for the enzyme product generated on the surface of the first phage will form a complex, leading to the proximity enhancement effect on infection of an indicator organism,

In a modification of this embodiment, the peptide sequence or protein may alternatively be coupled directly to the surface of the virus using standard chemical coupling techniques. Other moieties may also be coupled to the virus surface, including lipids, phospholipids, sugar residues and oligonucleotides. In all these cases the underlying mechanism is identical: enzyme reaction followed by formation of a complex involving further viruses conjugated to specific binding agents and the detection of the complex through a proximity enhancement effect on infection of an indicator organism.

Using a random peptide sequence expressed or coupled on the surface of a virus it is possible to explore the specificity of an enzyme that modifies the sequence as only the viruses with modified peptides will form the subsequent complex and so will produce a dual infection of the bacterial cell. The sequence of the expressed peptides can then be determined by sequencing the viral nucleic acid after culturing the indicator organism.

The multiple virus method can be used as a quantitative assay for the activity of an enzyme such as a kinase since the enzyme will generate a product moiety that promotes the close approach of two or more virus linked conjugates and their subsequent infection of an indicator organism. The extent or rate of growth of the organism is a measure of the activity of the enzyme.

The multiple phage enzyme assay method can be homogenous (no separation step) or heterogeneous (with separation step). For homogeneous assays the binding interaction between the phages and the product of the enzyme reaction is carried out in solution in, for example, the wells of a 96, 384 or 1536 well micro plate. Alternatively one of the phages can be immobilised to the surface of the microwell. On completion of the incubation with two or more phages, an excess of indicator organism is added to the well and the infection takes place in solution. The final step in the procedure is the detection of the multiply antibiotic resistant bacterial cells in the well of the microplate. For heterogeneous assays one or more of the phages are bound or become bound to a solid phase, which could be a microwell or a magnetic particle, and after the incubation where the complex forms between phages and the enzyme product the solid phase is washed to remove reactants. After the wash step the indicator organism is added to the well and infection takes place on the surface of the solid phase. Again the final step in the procedure is the detection of the multiply antibiotic resistant bacterial cells in the well of the microplate.

Further examples of methods according to the invention include the following. In one type of method, said first reactant is a substrate and the second reactant is a reagent for cleaving the substrate, and the method comprises exposing the substrate to the reagent for cleaving said substrate to form a first and a second cleavage product, wherein said substrate has a first binding site for binding a first virus and a second binding site for binding a second virus and said first and second binding sites are separated from one another by said cleavage reaction,

before or after said cleavage reaction, exposing said substrate or said first and second cleavage products to at least said first virus and said second virus under conditions such that if intact substrate is present the two viruses will bind to their respective binding sites to form a multiple-virus tagged complex, and

detecting or quantitating the presence of said complex after said cleavage reaction.

In another preferred method, said first reactant is a mixture of a first substrate and a second substrate and said second reactant is a reagent for joining said first and second substrates to for a ligated product, said method comprising:
exposing said first substrate and said second substrate to said reagent to join said substrates to form a ligated product, wherein said ligated product has a first binding site for binding a first virus and has a second binding site for binding a second virus which said first and second binding sites are not both present in said first and second substrates,
before or after said ligating reaction, exposing said ligated product or said first and second substrates to at least said first and second virus under conditions such that the two viruses will bind to their respective binding sites and in the ligated product will form a multiple-virus tagged complex, and
detecting or quantitating the presence of said complex after said ligating reaction.

The multiple phage enzyme assay is a general assay method for any enzyme reaction that involves the synthesis or degradation of a substrate that can modulate the proximity, or close approach, of two or more phages that are conjugated to specific binding agents that recognise the same or different moieties on the substrate. Close approach of the phage conjugates results in a marked increase in the efficiency of multiple infection of a bacterial cell.

Enzymes that add or remove specific groups to substrates can all be assayed using the multiple phage technique. Possible enzymes include:
kinases—add a phosphate group to tyrosine, threonine or serine residues, leading to activation or inactivation of enzyme activity
phosphatases—remove a phosphate group from tyrosine, threonine or serine residues, leading to activation or inactivation of enzyme activity
adenylation enzymes
ubiquitinylation enzymes
proteases—cleave peptide and protein sequences at specific sites
esterases
ligases
polymerases
nucleases
methylases
glycosidases and other enzymes that modify sugars

The invention is not limited however to embodiments in which the second reactant is an enzyme.

The invention includes an assay kit for assaying the outcome of a reagent-substrate reaction, comprising as a first reactant a substrate for a reagent, and as a second reactant a said reagent which cleaves or extends said substrate, a first virus tag capable of binding or bound to said substrate and a second virus tag capable of binding said substrate before but not after cleavage thereof or capable of binding said substrate after but not before extension thereof, to form a multiple-virus tagged complex comprising said first and second virus tags and said non-cleaved or extended substrate. Such a kit includes an assay kit for assaying the effectiveness of a modulator of a reaction between a first reactant and a second reactant, comprising a said first reactant, a first virus tag bound to said first reactant or for binding to said first reactant, a said second reactant for reaction with said first reactant to form a reaction product, and a second virus tag for binding to the reaction product of said first and second reactants, wherein said second virus tag does not bind to said first or second reactant prior to reaction thereof.

The invention further includes an assay kit for assaying the effectiveness of a modulator of an enzyme-enzyme substrate reaction, comprising an enzyme substrate, a first virus tag bound to said enzyme substrate or for binding to said enzyme substrate, an enzyme for reaction with said substrate to form a reaction product, and a second virus tag for binding to the reaction product of said enzyme and enzyme substrate, wherein said second virus tag does not bind to said substrate prior to reaction thereof with said enzyme. In either of these embodiments the kit may include at least one candidate modulator for said reaction.

Apart from the screening of modulators, the method of the invention can be used to assess the presence and the approximate amount or concentration of the first reactant or the second reactant in a sample or of the presence, amount or effectiveness of a reaction modulator and thus may be carried out to give a quantitative assessment of any of these materials as well as to detect simply their presence or otherwise. In some instances, it may be possible to monitor the generation of one of these materials in a sample by monitoring the development of some secondary feature, for example colour, associated with the presence of the multiple-virus tagged complex. The determination of the presence or amount of the multiple-virus tagged complex may be carried out intermittently or continuously over a period of time or as a single observation.

Where the determination of the presence or amount of the multiple-virus tagged complex is carried out using a bacterium as an indicator material, the viral moieties may attach to the indicator material in a number of ways. For example, the viral moieties may infect bacterial indicator materials; or may express a gene expression product during the cultivation which attaches to the indicator material to impart the distinctive properties thereto. However, the transfer of the characteristic properties from the multiple-virus tagged complex to the indicator material need not require the entry of the viral particles into the cell of the indicator material. Thus, the virus particles may transfer their properties by becoming attached to the exterior of the indicator material.

For convenience, the invention will be described hereinafter in terms of the case where the viral moieties carried by the target material infect the bacterial cells in the detection step and the viral DNA is transcribed within the infected cells to impart the distinctive properties to the infected cells.

Thus, the detection stage may be carried out by adding an appropriate bacterial culture to the multiple-virus tagged complex and carrying out the cultivation and infection of the bacteria under conditions in which only those bacteria having both distinctive properties imparted to them survive or are able to replicate. Thus, in the preferred embodiment, cultivation of bacteria such as E. coli is carried out in the presence of the two anti-bacterial agents to which the viral moieties carried by the virally bound target material impart resistance. Alternatively, the cultivation is carried out in the absence of the antibiotics and the antibiotics are added after the cultivation has been carried out to provide a readily detectable bacteria population. For convenience, the invention will be described hereinafter in terms of carrying out the cultivation of the bacteria in the presence of the antibiotics so that their effect on the growth of the bacterial cells can be detected in real time.

The E. coli is preferably present in a greater amount than would be required to achieve numerical parity between the virally bound target molecules or particles and the bacterial cells, preferably in excess of the statistically required amount. The use of an excess of the E. coli reduces the chance that an individual bacterium cell will be infected by individual particles of both viruses which may remain in the product from earlier in the process. However, where the conditions in the detection stage or subsequently are strongly adverse to the survival or growth of cells infected by only one of the types of virus, it may not be necessary to use an excess of the E. coli. Typically, an excess of from 10 to 300% or more of the E. coli over the statistical amount will be used to minimise the unintended production of bacteria cells which are infected with both types of virus through multiple independent infection events each involving infection with only one type of virus and thus aid detection of the cells infected in a dual infection event. Furthermore, as stated above, it is preferred to remove unbound virus particles from the multiple-virus tagged complex, which further reduces the risk of such unintended production of infected bacteria cells.

Depending upon the efficacy of the antibiotic agent used in the detection stage, the bacterial cells infected by only one virus may die or may not grow and replicate. If lysogenic viruses are used, the dually infected cells can host viral replication. The replicated viral particles from such cells can therefore infect further cells and replicate so that a cascade of infection, replication and release of viral particles can be caused. This has the effect of amplifying the effect of the growth of the dually infected cells.

The growth of the dually infected cells can be monitored using any suitable technique. For example, cultivation of the bacteria can be continued until significant populations of the dually infected cells are apparent to the naked eye. Alternatively, where one of the properties imparted to the target material permits it or where a third virus has been bound to the target material, the growth of the dually infected cells can be monitored by colourimetric, fluorescent or luminescent means. If desired, the cultivation means can incorporate an enzyme or other means which responds to a chemical released from the growing bacteria to enhance the property endowed by one of the viruses carried by the dually infected cells.

The distinctive property imparted to the dually infected bacterial cells varies in intensity according to the number of such cells present in the cultivation mixture. This intensity can be used to give an indication of the amount of a reactant in the original sample or the complex as well as showing that the reactant or a modulator was present in the initial sample tested.

If desired, the effect of background coupling of individual phage particles in the multiple-virus tagged complex and their subsequent detection can be reduced using a surfactant, notably a non-ionic surfactant such as that sold under the trade mark Tween or by the use of a protein blocking agent such as casein or albumen in amounts of from 0.1 to 0.5% v/v and up to 5% w/v respectively in the cultivation medium or washing fluids.

By way of illustration, two different viruses, virus A and virus B are used and each is a single-stranded bacteriophage M13 virus or phagemid modified by insertion of the genes encoding ampicillin or kanamycin resistance respectively. Other antibiotic resistances such as chloramphenicol resistance could of course be used. If desired, the viruses can be modified by insertion of the gene encoding the IgG binding domain of the proteins A or G. This enables the viruses to be bound to any IgG antibody which can be specific for the target material. Where the target molecule is in solution, either or both viruses A and B can be further modified to include a maltose binding peptide or auto-biotinylation peptide or covalently linked to a hapten such as biotin. This enables the multiple-virus tagged complex to be washed by capture with streptavidin or maltose-derivatised paramagnetic beads in order to remove any unbound virus. This increases the sensitivity and specificity of the method of the present invention.

Virus A and/or B can also be modified to contain a gene which encodes a detectable marker, such as an enzyme for example β-galactosidase or luciferase, which enables colorimetric, fluorescent or luminescent detection of the multiple-virus tagged complex. If desired virus A and virus B may each contain different components of the detectable marker which are complemented and become functional upon dual infection of the indicator material. A specific example of this is the expression of β-galactosidase following LacZ complementation of TG1 cells. The expression of the β-galactoaidase only occurs upon infection of the TG1 cells with a multiple-virus tagged complex carrying both the complementary components of the lac operon. The β-galactosidase can be detected using the inducer isopropyl-β-thiogalactopyranoside and the indicator bromo-4-chloro-3-indolyl-β-galactoside in an agar medium. The medium may contain 4-methylumbelliferyl-β-D-galactose which is fluorescent or luminescent in the presence of β-galacotsidase. It is also within the scope of the invention to monitor the changes in colour or colour intensity intermittently or continuously using a spectrometer, ELISA reader, luminometer or fluorimeter.

Virus particles may be incubated for 10-120 minutes at a temperature between 4° C. and 50° C. with the molecule to which they are to bind. Once the multiple-virus tagged complex has been formed, the reaction mixture may be either used as such in a bacterial cultivation stage, or the complex may be isolated, for example using bead capture and may be washed. Where the complex is bound to a solid support when it is formed or thereafter, it can be rinsed with an appropriate wash buffer.

In a preferred bacterial cultivation stage, an excess of Escherichia coli (E. coli) over the statistically required amount is then added in an appropriate growth media and incubated at a temperature between 4° C. and 50° C. for 30-720 minutes. The growth medium contains both ampicillin and kanamycin so that bacteria which have been infected by only one of the viruses die or do not replicate, whereas those which have been infected by both viruses A and B are resistant to these antibiotics and replicate. This enables an observer to monitor the growth of the bacterial cells in the culture medium and to determine both the presence of growing cells and the number of such cells in real time. However, it is also possible to add the antibiotics to the culture medium after a suitable incubation period and to determine the effect of the anti-biotic on the cell population. If the antibiotic is added after the initial incubation stage, a further incubation of between 2-60 minutes would be required in order to generate a detectable change in the incubated material.

In another preferred embodiment for the detection of a reaction product molecule such as a nucleic acid, which may be in solution or bound to a solid support, virus A and virus B are single-stranded bacteriophage M13 viruses or phagemids which have been modified by insertion of the genes encoding ampicillin and kanamycin resistance respectively. The viruses may be subjected to further modification by linking to nucleic acid or peptide nucleic acid probes as described below. The viruses are incubated for 30-240 minutes at a temperature between 4° C. and 60° C. with their target material (first reactant or reaction product). During this incubation, the viruses bind to their target nucleic acid molecules in the sample.

Here, the reaction to be monitored may be the reaction of a nucleic acid first reactant with a nuclease, a polymerase, or a ligase producing a shorter or longer nucleic acid.

After this incubation, the doubly virally bound nucleic acid reaction product is incubated with an excess of E. coli in a suitable growth medium at a temperature between 4° C. and 50° C. for 30-480 minutes and the effect of the antibiotics assessed as described above.

In the detection of nucleic acid reactions, two or more nucleic acids probes are required which can bind to the target nucleic acid materials. If two probes are used they can be labelled with the same or differing haptens eg. both probes can be labelled with biotin or one probe labelled with biotin and the other with digoxigenin (Roche. Lewes, UK). After binding (hybridising) the probes to the nucleic acid reaction product via features present in the starting nucleic acid first reactant and introduced in the course of the reaction respectively, the probe molecules which are now spatially linked can be detected through the hapten groups carried by them. For example, Phage A and Phage K can be used which have been conjugated to fragments of anti-digoxigenin antibody (Roche, Lewes, UK) or streptavidin. If a single hapten, eg. biotin, is used to label the probes, then the two phage can be labelled with the same hapten-binding molecule, eg streptavidin. This approach may be adequate where the first phage is bound to the first reactant before the reaction. The target DNA can be prepared from the organism using a suitable conventional technique or in crude extracts or lysates of patient samples and immobilised onto a membrane eg. Hybond N, Hybond N+ (Amersham International plc, Amersham UK) and denatured using standardised methods (Amersham International plc publications P1/384/91/6 and P1/387/92/4 and Short Protocols in Molecular Biology, second edition. Ausubel, F M et al eds. Green Publishing Associates and John Wiley & Sons. 1992) prior to detection. Alternatively, a homogenous assay format can be used; or purified DNA can be prepared, cut by restriction enzymes, size separated by electrophoresis and immobilised onto nylon membranes before reaction.

The invention will be further described and illustrated by the following Examples in which reference is made to the accompanying drawings in which:

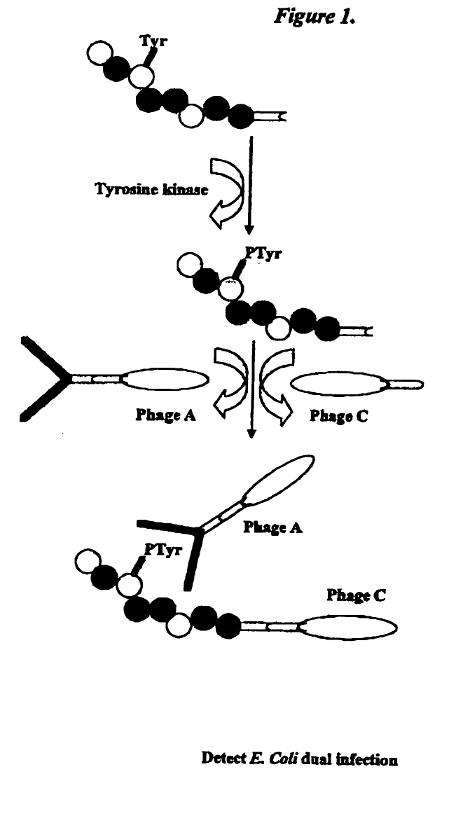

FIG. 1 shows a first reaction scheme;

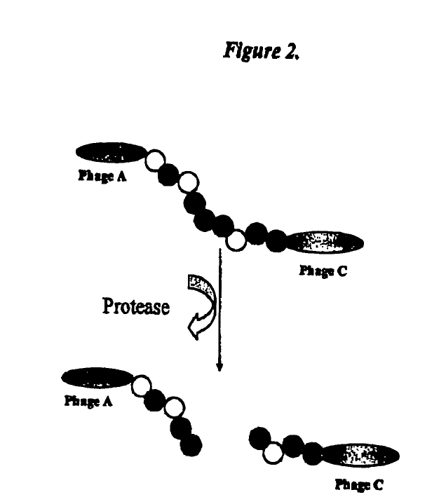

FIG. 2 shows a second reaction scheme;

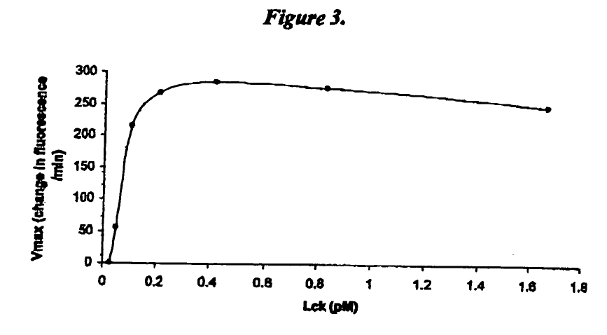

FIG. 3 shows results obtained in Example 1;

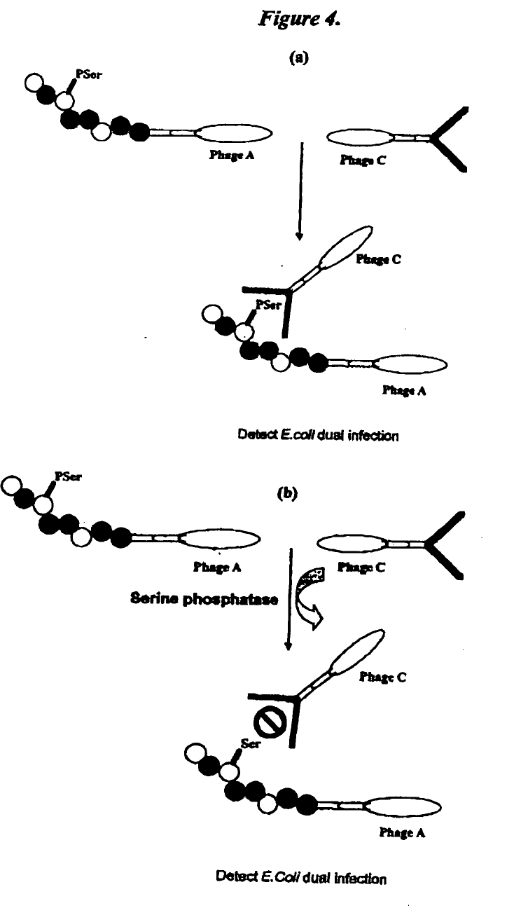

FIG. 4 shows a third reaction scheme; and

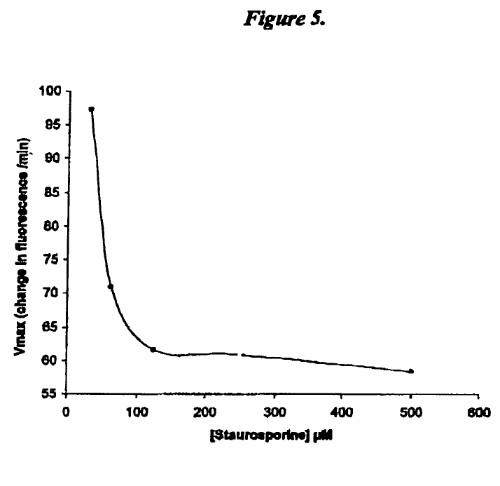

FIG. 5 shows results obtained in Example 4.

### EXAMPLE 1

### Tyrosine Kinase Essay

Assay Principles

FIG. 1 shows a schematic diagram of the tyrosine kinase assay principles. The assay uses a tyrosine containing synthetic peptide substrate that is biotinylated at the amino terminus. In the presence of a tyrosine kinase the tyrosine residue is phosphorylated. Anti-phosphotyrosine antibody coupled to phage A and streptavadin conjugated to phage C are added to the reaction. Phage A and C code for ampicillin and chloramphenicol resistance respectively. Anti-phospho-tyrosine antibody—phage A binds to the newly formed phosphate group and the streptavadin—phage C binds to the biotin moiety of the substrate. Thus both phage A and C are brought in close proximity of each other. Addition of E. Coli (in the presence of ampicillin and chloramphenicol) leads to a rapid dual infection of the E. Coli by phages A and C. Only phages within close proximity of each other will generate dual infected E. Coli (these are identified by their resistance to ampicillin and chloramphenicol). The numbers of viable E. Coli are enumerated via a number of different techniques depending upon the nature of the application. For drug discovery applications the E. Coli is encoded with a β-lactamase, which is detected, using a fluorescent substrate.

Assay Protocol

Materials and Instrumentation

Assay buffer—50 mM Hepes (pH7.0), 10 mM MgCl₂, 0.1% BSA and 1 mM DTT.
Protein Kinase—100 ng/ml stock solution (Sigma).
Substrate—the substrate mixture consisting of 100 μM ATP and 2 μM N-biotinylated peptide substrate (Pierce) were prepared in assay buffer
Reagent mixture—a reagent cocktail consisting of the following items is prepared in assay buffer and stored in the dark at 4° C.:
   Biotinyl anti-phosphotyrosine kinase antibody—streptavidin phage C—500 nM (antibody obtained from Sigma).
   Streptavidin-phage A—500 nM.
   12 μg/ml of ampicillin
   12 μg/ml of chloramphenicol.
E. Coli—log or less preferably stationary phase culture (stored at 4° C. until use).
β lactamase substrate—CCF2/FA (Aurora Biosciences Corp).
96 well plate flurometric reader—SpectraMax Gemini (Molecular Devices).
   Protocol

50 μl of the substrate mixture is added to 96 well black plates and the kinase reaction is initiated by the addition of 40 pM of enzyme. Following a 30-minute incubation at room temperature, 150 μl of the reagent mixture are added to the reaction mixture. The reaction mixture is left to incubate at room temperature on a plate shaker for 30 minutes. 20 μl of log (or less preferably stationary) phase E. Coli are then added to the reaction and left to incubate at 37° C. for 15 minutes. 10 μl of CCF2/FA is then added to the reaction mixture and the plate is read with the SpectraMax in the kinetic mode over a period of 30 minutes.

Following the principles outlined above, we have developed a highly sensitive homogeneous assay for lck kinase (p56^{lck}). p56^{lck} kinase is a membrane-associated non-receptor tyrosine kinase that is found exclusively in natural killer (TK) cells and T-cells (1) that play a critical role in T-cell development and activation. The p56^{lck} kinase is localised to a site on the genome that frequently contains chromosomal abnormalities in lymphomas and neuroblastomas (2). In the light of these observations, inhibitors for p56^{lck} kinase could have important applications in the treatment of autoimmune and cancer disease.

The kinase substrate peptide (poly-Glu-Tyr-biotin, Pierce, USA) was coupled to streptavidin derivatised M13 (encoding for ampicillin resistance) using standard biotin-streptavidin conjugation techniques (3). Biotinylated anti-phosphotyrosine antibody (Sigma, USA) was coupled to streptavidin derivatised M13 bacteriophage encoding for chloramphenicol resistance. These phage conjugates were purified using an affinity column containing anti-M13 antibody (Sigma Chemical Co.) which was bound to agarose. Using the Sigma protein tyrosine kinase assay kit (non radioactive) we determined that each phage carried approximately 10-100 ligands.

Serial dilutions of p56^{lck} kinase (Upstate, USA) were prepared in kinase buffer (1% BSA, 20 mM HEPES (pH7.4), 10 mM MgCl₂, 100 μM CaCl₂), 10 μl of each dilution was placed in a flat-bottom black microtitre plate together with 10 μM ATP. 10 μl of phage C-peptide substrate (10⁵ virons) conjugate were incubated with the kinase for 30 minutes at room temperature. 10 μl of phage A-antiphosphotyrosine antibody conjugate (10⁵ virons) was then added to the reaction and left to incubate for 30 minutes at room temperature. Next, 200 μl of log phase culture of E. coli (approximately 5×10⁷ cells) were added and incubated at 37° C. for 5 minutes. 5 μl (5 μM) of C₁₂-resazurin (Molecular Probes, USA) 10 μl of ampicillin and chloramphenicol (10 μg of each) were added to the reactions. The plate was covered with a transparent ‘breathable’ plate seal (Nalge Nunc, USA) and the change in fluorescence (excitation/emission at 530/590 nm) per minute was recorded over a period of four hours (Vmax) using a plate reader. FIG. 3 shows the results from the lck kinase dilution curve.

In the dual phage assay incubation the optimal number of each phage was determined to be 10⁵ virions. Previous optimisation experiments had shown that using a lower phage concentration decreased the signal and increased the detection time, whilst using a higher concentration led to an increase in the background signal. At the optimal concentration of phage the signal to background ratio of the Dual Phage technology was >10:1. In replicate p56^{lck} kinase assays, the CV was 5.1% (N=10).

The dual phage lck kinase assay has a lower detection limit (0.05 pmol of lck kinase/L) than other homogeneous lck kinase assays. For example the Packard HTRF lck assay has a sensitivity of 2 pmol/L (4). The homogeneous nature of the dual phage assay makes it ideally suited to both automation and miniaturisation. The labelled phages and the indicator organism are extremely robust (no loss of activity has been seen in phage conjugates and freeze dried E. coli stored at 4° C. over a period of six months) and can be readily prepared using standard techniques.

### Example 2

### Protease Assay

Assay Principles

The dual phage technology can be utilized in different configurations to assay for proteases. FIG. 2 shows a schematic of one way in which the dual phage system is used to detect protease activity. Phage A and C are coupled to the terminal amino groups of a synthetic peptide. In the absence of a protease the peptide will not be cleaved thus the phage A and C will remain in close proximity of each other leading to a successful dual infection of E. coli. In the presence of a protease the peptide will be cleaved and Phage A and C will be separated thus dual infection of E. Coli will not be observed.

Materials and Instrumentation

Peptide substrate—a synthetic peptide labelled at each terminal amino group with phage A and C.
E. coli—log or less preferably stationary phase culture (stored at 4° C. until use)
Antibiotics
   12 μg/ml of ampicillin
   12 μg/ml of chloramphenicol.
β lactamase substrate—CCF2/FA (Aurora Biosciences Corp).
Fluorometric 96 well plate reader—SpectraMax Gemini from Molecular Devices.
   Protocol
   Control Reaction

20 μl of E. coli are added to 200 μl of peptide substrate. The reaction mixture is left to incubate at 37° C. for 15 minutes. 10 μl of the CCF2/FA is then added to the reaction mixture and the plate is read with the SpectraMax in the kinetic mode over a period of 30 minutes.

Protease Sample

20 μl of protease is added to 200 μl of the peptide substrate. The reaction is left to proceed for 30 minutes at room temperature. 20 μl of log (or less preferably) stationary phase E. coli is then added to the reaction and left to incubate at 37° C. for 15 minutes. 10 μl of CCF2/FA is added to the reaction mixture and the plate is read with the SpectraMax in the kinetic mode over a period of 30 minutes.

### Example 3

### Phosphatase Assay

Assay Principle

An example of a dual phage based phosphatase assay was set up using serine phosphatase as model enzyme system. The reaction scheme involved is illustrated in FIG. 4, panels (a) and (b). The assay is based on the principle of inhibition of a dual phage complex formation. In the absence of serine phosphatase (panel (a)) phages A and C (labelled with a phosphopeptide and an antiphosphoserine peptide antibody respectively) will form a dual phage complex. However when there is serine phosphatase activity (panel (b)), the phosphopeptide on phage A will be dephosphorylated and a dual phage complex will not form.

Protocol

Phosphatase Reaction

1. Serial dilutions of serine phosphatase were prepared in phosphatase buffer (1% BSA, 20 mM HEPES (pH7.4) and 1 mM MgCl₂).
2. 10 μl of each dilution was placed in a flat bottom black microtitre plate.
3. 10 μl of phage A-phosphopeptide substrate (10⁵ virions) conjugate was added to the phosphatase dilutions and incubated for 30 minutes at room temperature.
4. 10 μl of phage C-antiphosphoserine antibody conjugate (10⁵ virions) was then added to the reaction and left to incubate for 30 minutes at room temperature.
5. Next, 20 μl of log phase culture of E. coli were added and incubated at 37° C. for 10 minutes.
6. 150 μl of LB broth containing 10 μg each of ampicillin and chloramphenicol were added to the reactions and incubated for 1 hour at 37° C.
7. 1 μl of C₁₂-resazurin (10 μg) was added to each well and the change in fluorescence (ex 530 nm and em 590 nm) was monitored over a period of 24 hours.
   Control Reaction

The control reaction (no phosphatase control) was performed as above but with the omission of steps 1 and 2.

Data Analysis

Data analysis was performed by determining the difference in the rate of change in fluorescence (per second) for the phosphatase and control reactions. It was observed that the rate of change of fluorescence in the microtitre wells was reduced in the presence of phosphatase and was inversely proportional to the amount of phosphatase in the well.

### EXAMPLE 4

### Assay for Modulation of of an Enzyme Reaction Detected by the Dual Phage Approach—Inhibition of Lck Kinase by Staurosporine

Assay Protocol

1. 10 μl of lck kinase containing 6.25⁻³ U (prepared in kinase buffer (1% BSA, 20 mM HEPES (pH7.4), 10 mN MgCl₂, 100 μM CaCl₂)) were incubated with various dilutions of staurosporine for 15 minutes at room temperature.
2. 10 μl mixture of 10 μM ATP and phage C-peptide substrate (10⁵ virions) complex were incubated with the kinase/staurosporine for 30 minutes at room temperature.
3. 10 μl of phage A-antiphosphotyrosine antibody complex (10⁵ virions) was then added to the reaction and left to incubate for 30 minutes at room temperature.
4. Next, 200 μl of log phase culture of E. coli were added and incubated at 37° C. for 5 minutes. 8 μl of an assay mix containing 5 μM C₁₂-resazurin (Molecular Probes, USA) fluorescent substrate, 1 μg of ampicillin and 1 μg chloramphenicol were added to the reactions. The plate was covered with a transparent ‘breathable’ plate seal and the fluorescence was monitored over a period of 4 hours.
   Results

The results are shown in FIG. 5 and show that the modulation of the activity of the lck kinase by the inhibitor staurosporine can be measured by the dual phage methodology.

Whilst the invention has been described chiefly in detail in terms of the infection of E. coli bacteria by E. coli infecting phage, it is stressed that the invention is broadly applicable through the use of any virus and any virus infected cell, including other bacteria and insect and mammalian cells and the viruses that infect them. Many other variations and modifications of the exemplified practice of the invention are possible within the general scope of the invention.

References

1. Veillette A, Abraham N, Caron L, Davidson D. The lymphocyte-specific tyrosine protein kinave p56lck. Sem Immunol 1991; 3:143-52.
2. Abraham K M, Levin S D, Marth J D, Forbush K A, Perlmutter R M. Thymic tumorigenesis induced by over expression of p56lck. Proc Natl Acad Sci U S A 1991; 1;88:3977-81,
3. Hemanson G T, ed. Bioconjugation Techniques, Academic Press 1996: 570-91.
4. Park Y W [Application note] Development and miniaturization of an HTRF tyrosine kinase assay, Packard Instrument Company, Meridian, Conn., Document No AN4002-DSC.

1. A method of assay for the outcome of a reaction comprising conducting a reaction between a first reactant and a second reactant to produce from the first reactant a product having at least one new structural feature not present in the first reactant or the second reactant, producing before or after said reaction a multiple-virus tagged complex comprising at least two different viruses and said product or said first reactant, in which complex at least one of said viruses is connected to said product using a said new structural feature or is lost from said first reactant upon formation of said new structural feature, and determining the presence or amount of said complex.

2. A method as claimed in claim 1, wherein the presence or amount of said complex is determined by a method comprising exposing to said complex an indicator material to which the viruses carried by the multiple-virus tagged complex attach so as to endow the indicator material with a distinctive property, and observing said distinctive property.

3. A method as claimed in claim 2, wherein said indicator material is a bacterial culture.

4. A method as claimed in claim 3, wherein the distinctive property is the ability to survive under specific culture conditions in which the bacterial culture without said distinctive property does not survive.

5. A method as claimed in claim 4, wherein said distinctive property is multiple antibiotic resistance.

6. A method as claimed in any one of claims 2 to 4, wherein the multiple-virus tagged complex endows the indicator material with a visually detectable feature.

7. A method as claimed in any one of claims 3 to 6, wherein said complex is cultured with at least the statistically required amount of bacterial cells.

8. A method as claimed in any preceding claim, wherein the second reactant is an enzyme.

9. A method as claimed in claim 8, wherein the enzyme is a kinase, phosphatase, adenylating enzyme, ubiqitinylating enzyme, protease, hydrolase, esterase, ligase, polymerase, nuclease, methylase, or glycosidase or other sugar modifying enzyme.

10. A method as claimed in claim 8 or claim 9, wherein the first reactant is a substrate cleaved by the enzyme.

11. A method as claimed in any one of claim 8 to 10, wherein the first reactant is a substrate to which a moiety is added by the enzyme.

12. A method an claimed in any preceding claim, wherein the first reactant is tagged with a virus prior to reaction with the second reactant and the multiple-virus tagged reaction complex is formed by exposing the product of the reaction between the first and second reactants to a second virus that binds to the said product to form the multiple-virus tagged complex.

13. A method as claimed in any one of claims 1 to 11, wherein the first reactant is not virus-tagged and wherein the product of the reaction between the first and second reactants is exposed to a first virus and to at least a second virus which each bind to the said product to form the multiple-virus tagged complex.

14. A method as claimed in any preceding claim, wherein the first reactant is a fusion protein or peptide expressed as part of a coat protein of a phage.

15. A method as claimed in any preceding claim, wherein the reaction between the first and second reactants is carried out in the presence of a modulator for said reaction.

16. A method as claimed in claim 1, wherein said first reactant is a substrate and the second reactant is a reactant for cleaving said substrate said method comprising:
exposing the substrate to the reagent for cleaving said substrate to form a first and a second cleavage product, wherein said substrate has a first binding site for binding first virus and a second binding site for binding a second virus and said first and second binding sites are separated from one another by said cleavage reaction,
before or after said cleavage reaction, exposing said substrate or said first and second cleavage products to at least said first virus and said second virus under conditions such that if intact substrate is present the two viruses will bind to their respective binding sites to form a multiple-virus tagged complex, and
detecting or quantitating the presence of said complex after said cleavage reaction.

17. A method as claimed in claim 16, wherein the reagent is an enzyme.

18. A method as claimed in claim 17, wherein the enzyme is a protease, hydrolase or nuclease.

19. A method as claimed in any one of claims 16 to 10, wherein the cleavage reaction is carried out in the presence of a modulator or candidate modulator of said cleavage reaction.

20. A method as claimed in claim 1, wherein said first reactant is a mixture of a first substrate and a second substrate and said second reactant is a reagent for joining said first and second substrates to form a ligated product, said method comprising:
exposing said first substrate and said second substrate to said reagent to join said substrates to form a ligated product, wherein said ligated product has a first binding site for binding a first virus and has a second binding site for binding a second virus which said first and second binding sites are not both present in said first and second substrates,
before or after said ligating reaction, exposing said ligated product or said first and second substrates to at least said first and second virus under conditions such that the two viruses will bind to their respective binding sites and in the ligated product will form a multiple-virus tagged complex, and
detecting or quantitating the presence of said complex after said ligating reaction.

21. A method as claimed in claim 20, wherein the reagent is an enzyme.

22. A method as claimed in claim 21, wherein the enzyme is a ligase.

23. A method as claimed in claim 22, wherein the first and second substrates are oligonucleotides.

24. A method as claimed in any one of claims 20 to 23, wherein the first and/or the second substrate is tagged with a virus prior to the ligating reaction.

25. A method as claimed in any one of claims 21 to 24, wherein the first and/or the second substrate is not virus-tagged and wherein the ligated product is exposed to form the multiple-virus tagged complex.

26. An assay kit for assaying the outcome of a reagent-substrate reaction, comprising as a first reactant a substrate for a reagent, and as a second reactant a said reagent which cleaves or extends said substrate, a first virus tag capable of binding or bound to said substrate and a second virus tag capable of binding said substrate before but not after cleavage thereof or capable of binding said substrate after but not before extension thereof, to form a multiple-virus tagged complex comprising said first and second virus tags and said non-cleaved or extended substrate.

27. An assay kit as claimed in claim 26 for assaying the effectiveness of a modulator of a reaction between a said first reactant and a said second reactant, comprising a said first reactant, a first virus tag bound to said first reactant or for binding to said first reactant, a said second reactant for reaction with said first reactant to form a reaction product, and a second virus tag for binding to the reaction product of said first and second reactants, wherein said second virus tag does not bind to said second reactant prior to reaction thereof with said first reactant.

28. An assay kit as claimed in claim 26 for assaying the effectiveness of a modulator of an enzyme-enzyme substrate reaction, comprising an enzyme substrate, a first virus tag bound to said enzyme substrate or binding to said enzyme substrate, an enzyme for reaction with said substrate to form a reaction product, and a second virus tag for binding to the reaction product of said enzyme and enzyme substrate, wherein said second virus tag does not bind to said substrate prior to reaction thereof with said enzyme.

29. An assay kit as claimed in any one of claims 26 to 28, further comprising at least one candidate modulator for said reaction.
